(19) United States
(12) Patent Application Publication
(10) Pub. No.: US 2021/0275307 A1
(43) Pub. Date: Sep. 9, 2021

(54) SURGICAL IMPLANT

(71) Applicant: IMPERIAL COLLEGE INNOVATIONS LIMITED, London (GB)

(72) Inventors: Jonathan Jeffers, London (GB); Richard Van Arkel, London (GB); Shaaz Ghouse, London (GB); Sebastian Ray, London (GB)

(73) Assignee: IMPERIAL COLLEGE INNOVATIONS LIMITED, London (GB)

(21) Appl. No.: 16/334,611

(22) PCT Filed: Sep. 20, 2017
(86) PCT No.: PCT/GB2017/052794

§ 371 Date: Mar. 19, 2019

(30) Foreign Application Priority Data
Sep. 20, 2016 (GB) 1616010.3

(51) Int. Cl. A61F 2/30

(52) U.S. Cl. CPC A61F 2/30771; A61F 2/3094; A61F 2002/30985; A61F 2002/30214; A61F 2002/30805; A61F 2002/30224; A61F 2002/30841

(57) ABSTRACT

A surgical implant (100) comprising a body having proximal and distal ends and a longitudinal axis extending there-between, the body comprising a core (105) and at least one end portion (115) at the distal end and a plurality of discrete whiskers (110) extending outwardly from the core (105) and at an acute angle relative to a longitudinal axis of the body in a proximal direction. The surgical implant preferentially allows direction in one direction and provides superior implant stability post-surgery due to the mechanical interaction between the whiskers and the bone structure providing increased resistance to pull-out of the implant.

This invention relates to a surgical implant, in particular a surgical implant which achieves enhanced mechanical fixation of a surgical implant to bone surface using mechanical whiskers on the implant.

### BACKGROUND

The global orthopaedic market is growing significantly due to the rise in prevalence of osteoarthritis propelling the demand for joint replacement surgeries. The ageing population, increased life expectancy and increased levels of obesity all contribute to the rise in osteoarthritis, and patients are less tolerant of living with the associated joint pain or constraint on their daily activities.

Joint replacements are predominantly performed in patients in their sixties or older, and surgeons are unwilling to perform this kind of surgery in younger patients due to the life expectancy of the replaced joint. Approximately 95% of these will last 10 years or more. However, revision is costly; the outcome is poor with a relatively high mortality risk. This is leading to alternative treatments of the arthritic disease progression where only parts of the joint are repaired rather than the whole joint replaced. Such partial joint replacements are smaller and fix to the bone over a smaller surface area than total joint replacements.

Implant loosening is one of the primary mechanisms of failure for hip, knee, ankle and shoulder arthroplasty, for partial or total joint replacement. Many established implant fixation surfaces exist to achieve implant stability and fixation, traditionally achieved through plasma spraying. More recently, additive manufacturing technology has provided new possibilities for implant design such as the provision of large, open, porous structures that could encourage bony ingrowth into the implant and improve long-term implant fixation.

U.S. Pat. No. 9,173,692 discloses composite metal and bone orthopaedic fixation devices that facilitate spine stabilization. These include a helical screw thread extending from a core to hold the device in position by friction. WO 2014/159216 likewise discloses a threaded bone anchor, this one having a reduced area open architecture thread profile. CN 204600649 discloses a barbed surgical nail used to fixate fracture fragments. The present application relates to an improved means of achieving mechanical fixation of a surgical implant.

### BRIEF SUMMARY OF THE DISCLOSURE

In accordance with a first aspect of the invention, there is provided a surgical implant comprising a body having proximal and distal ends and a longitudinal axis extending therebetween, the body comprising a core and at least one end portion at the distal end and a plurality of discrete whiskers extending outwardly from the core and at an acute angle relative to a longitudinal axis of the body in a proximal direction.

The acute angle of the whiskers relative to the longitudinal axis of the implant means that the whiskers flex towards the centre of the implant, away from the bone surface as the implant is inserted into the bone, yet flex outwardly, to grip onto the bone surface when the surgical implant is being urged out of the bone. An advantage of the whiskers deflecting away from the bone during insertion is that it reduces the resistive push-in load of the surgical implant and preserves more of the bone structure during surgery. However, it is the whiskers gripping onto the bone when the surgical implant is being pulled out that is particularly advantageous over conventional implant surfaces, as this mechanical interaction between the surgical implant and the bone increases the pull-out force required to dislodge the implant from its post-operative position and ensures greater stability of the implant. In order for the implant to become loosened, the whiskers would have to be deformed or broken, or the bone surface would have to be sheared.

In embodiments of the invention, the surgical implant may also comprise a distal end portion which tapers distally to facilitate insertion of implant into the bone. This has the additional advantage of avoiding sharp edges on the implant which would lead to increased stresses at the edge of the implant surface and result in the formation of fibrous tissue. Also, the tapered distal end aids the surgeon in inserting the implant into the associated prepared bone surface.

In embodiments of the invention, the surgical implant may comprise a substantially cylindrical body. A cylindrical body has the advantage of being a simple design that can be inserted directly into a pilot hole drilled into the bone. While a cylindrical body is advantageous, it would be within the scope of the invention for the surgical implant body to be of an irregular shape, which may depend on the geometry of the specific bone in which the surgical implant is to be implanted. Substantially cylindrical is intended to define a shape that has an overall impression of a cylinder, but which may include minor portions that are recessed or which are in relief of the cylindrical surface, and which may also have a surface texture that is not smooth. In particular, an implant including the above-described tapered distal end portion may still be considered as substantially cylindrical.

Alternatively, the whole surgical implant may taper distally. A tapering implant has the advantage of being easier to insert into a corresponding tapering hole in the bone than a straight-sided equivalent. In embodiments, the core has a constant cross-section perpendicular to the longitudinal axis and the whiskers are progressively shorter towards the distal end. Thus, the tapering shape may be provided by the progressive length of the whiskers along the implant, extending from a cylindrical core.

The surgical implant comprises whiskers which grip the bone in order to resist pull-out forces applied to the implant. To achieve sufficient stability in the bone, the arrangement, density and geometry of the whiskers may be optimised. The density of whiskers over a surface of the core may be in the range: 25 whiskers/cm² to 1000 whiskers/cm² and preferably 120 whiskers/cm² to 200 whiskers/cm². The whiskers may have an external length in the range: 0.7 mm to 12.0 mm and preferably 2.5 mm to 6.0 mm, as measured normal to the surface of the core from which the whiskers extend. The whiskers may form an angle with the longitudinal axis of the surgical implant in the range: 5° to 89° and preferably 20° to 60°. The whiskers may have a thickness in the range: 100 μm to 1000 μm and preferably 200 μm to 500 μm.

Some or all of the whiskers may be tapered in thickness along all or part of their length. Typically, the whiskers may be thicker at their root, where they are joined to the core, than at their free end. Alternatively or additionally, the whiskers may be tapered in thickness along the length of the implant, such that the whiskers at the distal end are progressively thinner than the whiskers at the proximal end.

Additionally, or instead of a taper, some or all of the whiskers may have a forked end. This particular embodiment would provide greater resistance to torsion of the surgical implant during insertion or post-surgery. The forked end may comprise a bifurcated end wherein each branch of the bifurcation lies in a plane outwardly offset from and substantially tangential to the surface of the core. This particular embodiment would provide greater resistance to torsion of the surgical implant during insertion or post-surgery.

The surgical implant may also comprise a core having a thickness in the range: 4% to 95% and preferably 36% to 75% of a total thickness of the implant, as measured normal to the longitudinal axis.

In embodiments, the core is tapered, narrowing towards the distal end of the implant. In conjunction with whiskers of common lengths, this arrangement will provide an implant having an overall distally narrowing taper. However, the lengths of the whiskers may progressively increase as the core narrows, such that the overall shape of the implant, defined by the free ends of the whiskers is substantially cylindrical.

Embodiments of the invention may comprise different whisker designs. The whiskers may include different cross-sectional profiles, such as square, triangular or circular profiles; different lengths or have a taper along their length. The whiskers may also comprise forked ends to further resist torsional motion of the implant during surgery. The forked end may comprise two branches that point in a proximal direction, but more than two branches would be possible, as would other variants of the forked end such as a cross or star-shaped pattern.

Embodiments of the invention may also comprise a porous structure in the form of a lattice structure surrounding the core of the surgical implant. This provides a relatively large, open, porous surface into which bony ingrowth is encouraged, further improving the long-term implant fixation. The lattice structure may have a depth in the range: 0.5 mm to 10 mm and preferably 2 mm to 5 mm, as measured normal to the surface of the core.

The lattice structure may have substantially the same outer perimeter as the outer perimeter of the distal end portion of the body, such that the outer surfaces of the lattice structure and the distal end portion of the body are contiguous with one another. In such an arrangement, the core would have a smaller perimeter or effective diameter than the distal end portion of the body, and the lattice structure would fill in the space outside the core defined by the outer extent of the distal end portion.

In embodiments of the present invention, the lattice structure may comprise a plurality of elements interconnected at nodes and forming voids with multiple vertices, such as triangular or quadrilateral voids. The voids may, instead of being two-dimensional, be three-dimensional, such as tetrahedral or cuboid voids.

The lattice structure may be formed by at least one layer of elements. Different layers may be overlaid, for example to improve strength in multiple directions.

The lattice may comprise one or more layers to form voids through which individual whiskers may protrude to mechanically engage with the bone. If the surgical implant has a pull-out force applied to it, the lattice may also be arranged so that elements interact with individual whiskers and provide further resistance to the whiskers deflecting, effectively shortening the length of those whiskers and thereby requiring greater force for a given deflection of the free end.

Hence, embodiments of the invention may comprise one or more whiskers extending through a respective void defined within the lattice structure. Further, the whiskers may be arranged to come into contact with a lower cage element defining the associated void when the whiskers are urged to deflect downwardly relative to the core, as would occur during application of a pull-out force on the implant. The whiskers may also be arranged to avoid contact with any cage element when the whiskers are urged to deflect upwardly relative to the core, such that the entire length of the whisker is free to deflect, thereby resulting in a minimal insertion force for the implant.

The whiskers may each extend beyond the lattice structure by a distance in the range: 0.2 mm to 2.0 mm, and preferably 0.5 mm to 1.0 mm, as measured normal to the surface of the core.

Embodiments of the invention may be manufactured by additive manufacturing or 3D printing techniques.

The surgical implant may be made from one or more of: titanium and alloys thereof, stainless steel, tantalum, and cobalt-chromium alloys.

The surgical implant may comprise a surgical anchor or a surgical peg, or may comprise all or part of a larger surgical implant—for example being a peg, keel or anchor-like structure projecting from a larger surgical implant.

The concept of whiskers extending from a base surface of a surgical implant at an acute angle relative to a normal the surface and in a direction to resist pull-out is itself considered to be inventive independently of the particular shape and configuration of the implant itself, so according to a second aspect of the invention, there is provided a surgical implant surface comprising a body having a plurality of discrete whiskers extending from the surface of the body, wherein an angle between each of one or more of the whiskers and a respective axis normal to the surface of the body originating from a corresponding joint between each respective whisker and the body is in the range: 5° to 89° and preferably 20° to 60°.

Preferred features of the second aspect correspond to those described above with respect to the first aspect of the invention, mutatis mutandis.

An important aspect of the invention is allowing for large deformation of the whiskers to take place, as this takes up the load being applied to the implant. However, directly loading the joint between the core and the root of the whisker should be avoided, to avoid excessive stresses causing the whiskers to be sheared off the core. Embodiments of the present invention solve this problem by bringing the joints between the whiskers and the core inside the lattice structure to avoid direct loading of the joint by the bone structure. This allows the whiskers to have sufficient length to deflect when the surgical implant has a pull-out force applied to it, while avoiding direct loading of the whisker joint by the bone structure. A further advantage of this embodiment is that by having the whiskers pass through the lattice structure, some or all of the whiskers will come into contact with the lattice structure when a pull-out force is applied, effectively shortening the length of whisker being loaded. This functional shortening of the whisker acts to stiffen the whisker, further resisting the pull-out load and improving the stability of the implant.

Embodiments of the invention may also comprise modifications of existing implant designs, such as components in replacement hips, shoulders, knees and ankles. Such components are anchored in the bone and traditionally secured by a combination of friction or bone screws or pegs. However, such fixation devices are have the disadvantage that screws can become undone or loosen over time and conventional implant surfaces treated with plasma spraying do not provide sufficient friction between the implant and the bone to ensure long term implant stability. Incorporating the present invention into these designs would provide a superior fixation technique that provides additional resistance to pull-out of the surgical implant.

### BRIEF DESCRIPTION OF THE DRAWINGS

Embodiments of the invention are further described hereinafter with reference to the accompanying drawings, in which:

FIG. 1 is a side view of a surgical implant according to an embodiment of the invention;

FIG. 2 shows a scanning electron microscopy (SEM) image of a portion of the surgical implant of FIG. 1;

FIG. 3 shows, in schematic form, a surgical implant according to an embodiment of the invention where the whiskers are tapered in thickness along their length;

FIG. 4 shows, in schematic form, a surgical implant according to an embodiment of the invention where the whiskers are cylindrical;

FIG. 5 shows, in schematic form, a surgical implant according to an embodiment of the invention where the whisker length tapers from the proximal end of the core to the distal end of the core;

FIG. 6 shows, in schematic form, a surgical implant according to an embodiment of the invention incorporating forked whiskers;

FIG. 7 shows, in schematic form, a side view of a surgical implant according to an embodiment of the invention where the thickness of the core tapers from its proximal end to its distal end;

FIG. 8 shows, in schematic form, a surgical implant according to an embodiment of the invention, where the core of the implant is itself a lattice structure;

FIG. 9 shows, in schematic form, a surgical implant according to an embodiment of the invention, where there is no lattice structure surrounding the core;

FIG. 10 shows an exemplary comparison of push-in and pull-out forces of the present invention against those of a conventional fixation device;

FIG. 11 shows the effect of varying the length of the whiskers has on the push-in and pull-out forces of an example of the present invention;

FIG. 12 shows the effect of varying the density of the whiskers has on the push-in and pull-out forces of an example of the present invention;

FIG. 13 shows the effect of having a lattice structure compared to not having a lattice structure has on the push-in and pull-out forces of an example of the present invention;

FIG. 14 shows the effect varying the interference has on push-in/pull-out forces for conventional devices;

FIG. 15 shows a surgical implant surface according to an embodiment of the invention, applied to the stem of a replacement hip;

FIG. 16 shows a surgical implant surface according to an embodiment of the invention, applied to the keel of a tibial tray;

FIG. 17 shows a surgical implant surface according to an embodiment of the invention, applied to the cup of an acetabular cup;

FIGS. 18A and 18B show a surgical implant surface according to an embodiment of the invention, applied to a wedge implant suitable for opening wedge osteotomies.

### DETAILED DESCRIPTION

The present invention provides a surgical implant that incorporates barb-like struts, or whiskers, that preferentially allow movement in one direction. The whiskers work by being able to flex away from the bone (effectively partially/fully ‘hiding’ within the implant boundaries) as the implant is inserted into the bone, whilst popping back out and gripping onto the bone when pulling the implant out of the bone thus requiring the whiskers to be deformed, or the bone sheared before the implant can be loosened. This results in a surgical implant that has a decreased push-in force and an increased pull-out force, and improves the initial stability of a surgical implant.

FIG. 1 is a side view of a surgical implant according to an embodiment of the invention. The surgical implant 100 comprises a body and a plurality of whiskers 110, the body comprising a core 105, a distal end portion 115 and a lattice structure 120 surrounding the core 105. Each of the whiskers 110 are discrete and originate from the core 105 of the surgical implant 100 and pass through an associated void 125 of the lattice structure 120. The distal end portion 115 may have a distal chamfer—i.e. it tapers distally, to facilitate insertion of the surgical implant 100 into a bone—for example into a reamed or drilled hole in the bone. However, the distal end portion 115 need not have a tapered edge. The lattice structure 120 has substantially the same outer extent as that of the distal end portion 115, such that the overall shape of the implant 100, as defined by the exterior surface of the lattice 120 and by the straight-sided part of the distal end portion 115, is cylindrical.

The distal end portion 115 may comprise part of the core 105 and the surrounding lattice structure 120, or may be a distinct part of the implant, albeit connected to the core 105.

The whiskers 110 are arranged to extend outwardly away from the core 105 in a proximal direction, at an acute angle relative to the longitudinal axis of the body. The longitudinal axis of the body extends between the proximal and distal ends of the body.

When not in use, the whiskers 110 are arranged to pass through the voids 125 of the lattice structure 120. As the surgical implant 100 is pressed into a bone, the whiskers 110 are arranged to deflect towards the proximal end of the body through the engagement of the free ends of the whiskers 110 with the bone surface (e.g. the interior of a hole prepared in the bone). The lattice structure 120 and whiskers 110 are arranged such that the lattice structure 120 does not prevent the whiskers 110 from deflecting towards the proximal end when the surgical implant 100 is being pushed into the bone. This is important, as allowing the whiskers 110 to deflect in an unobstructed manner during insertion allows the surgical implant 100 to be inserted with a reduced push-in force compared to conventional surgical devices. Reduced push-in forces reduce damage to the bone during insertion of the implant in surgery. Also, structural integrity of the bone is retained and subsequent implant stability is enhanced.

Once inserted into the bone, the whiskers 110 are engaged within the bone structure and provide mechanical resistance to being pulled out. This is achieved by the proximally-directed whiskers 110 opposing any pull-out forces applied to the surgical implant 100. When a pull-out force is applied to the surgical implant 100, the whiskers 110 are urged to deflect towards the distal end of the surgical implant 100. This distal deflection of the whiskers 110 due to contact with the bone surface results in a load being applied to the whiskers 110 which opposes the pull-out force being applied to the surgical implant 100.

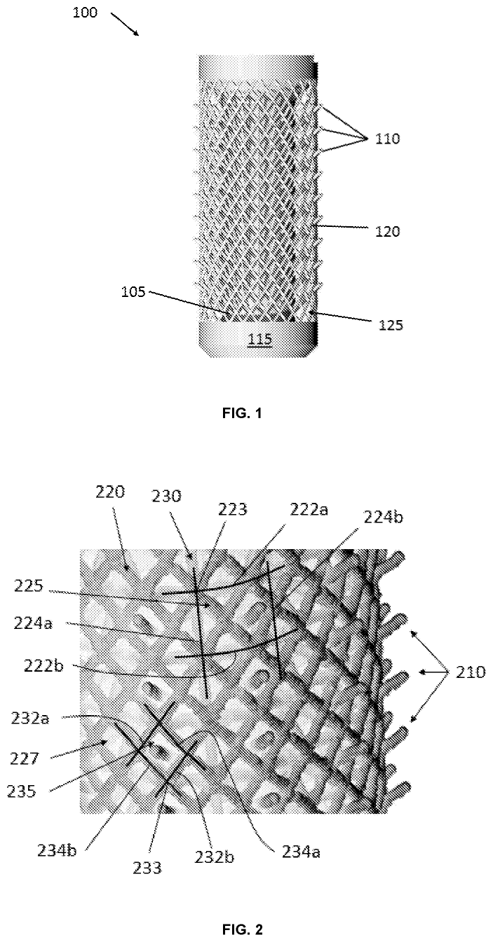

FIG. 2 shows a scanning electron microscopy (SEM) image of a lattice suitable for use with embodiments of a surgical implant according to the present invention. The SEM image generally shows a plurality of whiskers 210, an outer lattice structure 220 and an inner lattice structure 230. The inner lattice 230 and outer lattice 220 each comprise layers of the lattice structure 120 generally denoted 120 in FIG. 1. The outer lattice 220 is substantially cylindrical and is formed from a plurality of interconnecting elements 222a, 222b, 224a, 224b that are joined together at nodes 223. Voids 225 are defined between conjoined sets of the interconnecting elements. In the illustrated example, the outer lattice 220 is formed of a plurality of parallel, horizontally aligned, circumferential elements 222a, 222b, intersecting with a plurality of parallel, vertically aligned longitudinal elements 224a, 224b to form square voids 225 therebetween. In the illustrated example, element 222b forms a lower cage element for the void 225.

In the illustrated example, the outer lattice 220 is substantially cylindrical, concentric with the inner lattice 220, and is formed from a plurality of interconnecting elements 232a, 232b, 234a, 234b that are joined together at nodes 233. Voids 235 are defined between conjoined sets of the interconnecting elements. The outer lattice 220 is formed of a plurality of parallel, helical elements 232a, 232b aligned at a first angle to the vertical intersecting with a plurality of parallel, helical elements 234a, 234b aligned at a second angle to the vertical to form diamond-shaped voids 235 therebetween. In the illustrated example, elements 232b and 234b may each be considered as forming a lower cage element for the void 235. In particular, the node 233 where the two elements 232b and 234b join defines the lower-most part of the associated void 235.

The inner lattice 230 and the outer lattice 220 may be connected to one another, for example where respective nodes 223, 233 overlap. It will be understood that the elements comprising the lattice structure 120 may be arranged and interconnected in many different ways and that the voids may therefore take many other shapes.

Where the lattice structure 120 comprises layers each defining two-dimensional voids, three-dimensional voids 227 are formed at regular intervals in the lattice structure 120 where the two-dimensional voids 225, 235 interconnect. In some instances, elements of the inner lattice 230 and/or the outer lattice 220 may obstruct a particular void 227 and as such, no whisker 210 will be able to pass through that particular void. However, in instances where there is a clear void 227, a whisker 210 is able to pass from its root at a joint with the core 105, through a first void 225 in the inner lattice 230 and a second void 235 in the outer lattice 220 (i.e. together comprising a conjoined void 227) and protrude beyond the surface of the outer lattice 220. Where a whisker 210 passes through a void 227 in the lattice structure 120 and protrudes beyond the lattice structure 120, said whisker 210 will be considered to have an associated void 227 and at least one associated lower cage element 222b; 232b, 234b; 233.

In the embodiment shown in FIG. 2, the voids 227 are defined by intersecting 2-dimensional voids 225 and 235, each respectively defined by the inner and outer lattice structures. However, it will be appreciated that either or both of the inner and outer lattice structures 220, 230 may define 3-dimensional voids, such as tetrahedral voids. The skilled person would also consider other geometries depending on the requirements of the surgical implant 100. Similarly, although the embodiment of FIG. 2 shows two layers: an inner lattice layer 230 and an outer lattice layer 220, it would also be apparent to the skilled person to consider a lattice structure comprising more than two layers or just a single layer of elements, if required by the surgical implant. A single-layer option may be considered when a larger surgical implant may be undesirable, for example, if an implant is to be implanted into a small bone, or where a thicker lattice structure would not provide further structural stability of the implant or aid further bone in-growth.

In embodiments of the invention shown in FIGS. 1 and 2, if a pull-out force is applied to surgical implant 100, whiskers 210 will come into contact with lattice structure 120 at the respective associated lower cage element 222b; 232b, 234b; 233 and is thus effectively shortened and therefore stiffer, further increasing its resistance to the pull-out force being applied to surgical implant 100.

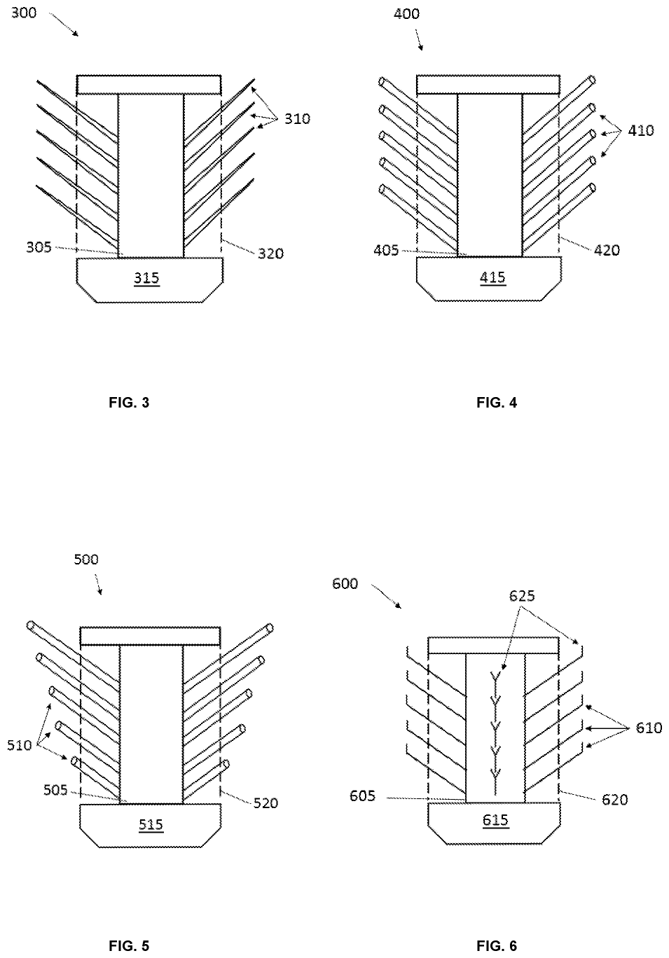

FIGS. 3 to 6 show different surgical implants according to different embodiments of the present invention where the whiskers are modified. FIG. 3 shows a surgical implant generally denoted 300 comprising a body having a proximal end and a distal end, the body comprising a core 305, a plurality of whiskers 310 extending from the core 305 in a proximal direction, and a distal end portion 315. A lattice structure 320 surrounds the core and has an outer extent that is contiguous with the distal end portion 315. The embodiment of the present invention shown in FIG. 3 shows whiskers 310 tapering in thickness along all of the whisker length from a root at the joint with the core 305 to a tip at the free end. The whiskers 310 may be tapered to a point, but it would be equally appreciated that the whiskers 310 may taper to an edge and have a triangular wedge-shaped profile, as is shown in FIG. 3. The embodiment of FIG. 3 shows a taper along the whole length of all of the whiskers. However, it would be equally appreciated that some or all of the whiskers 310 may not be tapered at all or some or that some whiskers may not be tapered along their entire length.

FIG. 4 shows a surgical implant generally denoted 400 comprising a body having proximal and distal ends, the body comprising a core 405, a plurality of whiskers 410 extending from the core 405 in a proximal direction, and a distal end portion 415. A lattice structure 420 surrounds the core and has an outer extent that is contiguous with the distal end portion 415. The whiskers 410 of this embodiment are uniform in thickness and in length. While straight whiskers of uniform thickness are shown in FIG. 4, the whiskers 410 may be configured to form small, hook-like structures (not shown). The hook-like whiskers may retain the directional bias of the other types of whiskers presently described.

FIG. 5 shows a surgical implant generally denoted 500 comprising a body having proximal and distal ends, the body comprising a core 505, a plurality of whiskers 510 extending from the core 505 in a proximal direction, and a distal end portion 515. A lattice structure 520 surrounds the core and has an outer extent that is contiguous with the distal end portion 515. The embodiment shown in FIG. 5 is substantially the same as that of FIG. 4, with the modification of the whiskers 510 of this embodiment tapering in length along the length of the implant, from the proximal end of the core 505 to the distal end of the core 505. Whiskers 510 of uniform thickness are shown in this embodiment, but it would be understood that other shapes, such as tapered, are envisaged.

FIG. 6 is a side view of a surgical implant according to an embodiment of the invention incorporating forked whiskers. The surgical implant in this embodiment is generally denoted 600 and is substantially the same as that in FIG. 1. The surgical implant 600 comprises a body having a proximal and distal end, the body comprising a core 605, a plurality of whiskers 610 extending from core 605 in a proximal direction, and a distal end portion 615. A lattice structure 620 surrounds the core and has an outer extent that is contiguous with the distal end portion 615. The whiskers 610 include a forked end 625. The forked ends 625 of the whiskers 610 are designed to resist rotational movement when torsional loads are applied to surgical implant 600. Each branch of the bifurcated forked end 625 lies in a plane outwardly offset from and substantially tangential to the surface of the core 605. The embodiment shown in FIG. 6 shows a forked end 625 with two branches, but it would be apparent to the skilled person that more branches could be included on each whisker 610 and that all whiskers 610 need not have the same number of forked ends 625.

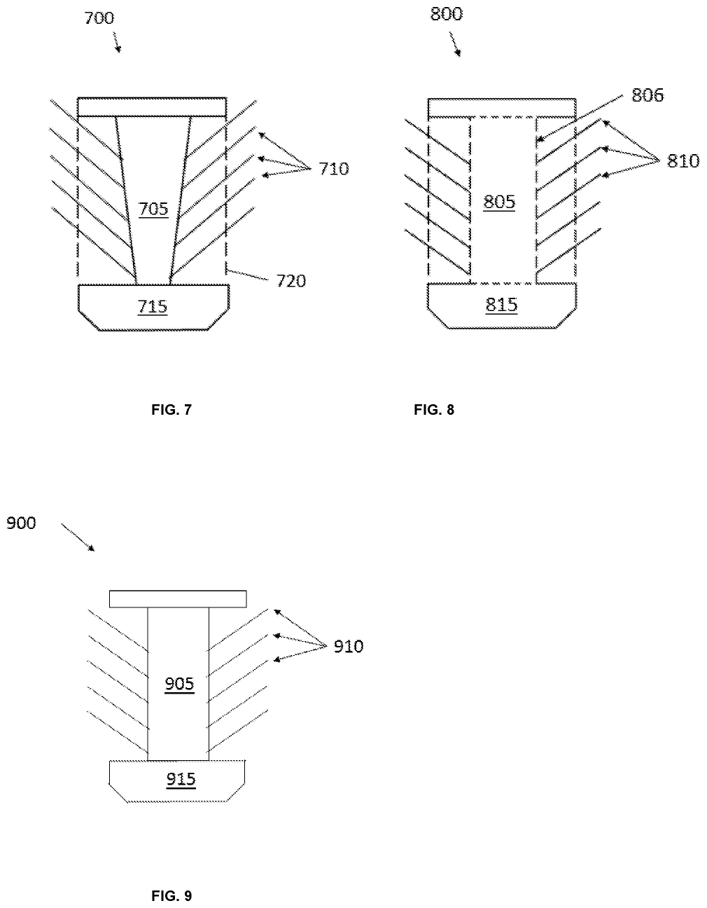

FIG. 7 shows a side view of a surgical implant generally denoted 700 according to an embodiment of the invention. The surgical implant 700 comprises a body having a proximal and distal end, the body comprising a core 705, a plurality of whiskers 710 extending from the core 705 in a proximal direction, and a distal end portion 715. A lattice structure 720 surrounds the core and has an outer extent that is contiguous with the distal end portion 715. The core 705 tapers in thickness from its proximal end to its distal end. The main advantage of this embodiment is that a reduced core thickness allows for longer whiskers 710 towards the distal end of the core 705, providing greater capacity to resist pull-out of the surgical implant 700, without requiring any increase in the overall size of the surgical implant 700.

FIG. 8 shows a side view of a surgical implant generally denoted 800 according to an embodiment of the invention, where the core 805 of the implant comprises a core lattice structure 806, which may be distinct from or integral with the lattice structure 120 described above, here illustrated as 820 and having an outer extent that is contiguous with the distal end portion 815. A plurality of whiskers 810 extend from an outer extent of the core lattice structure 806 in a proximal direction. With a lattice structure 806 forming the core, the entire implant can be constructed to have a stiffness matching that of the surrounding bone, thereby mitigating against stress-shielding. Also, the fully porous structure that results may provide improved bone ingrowth.

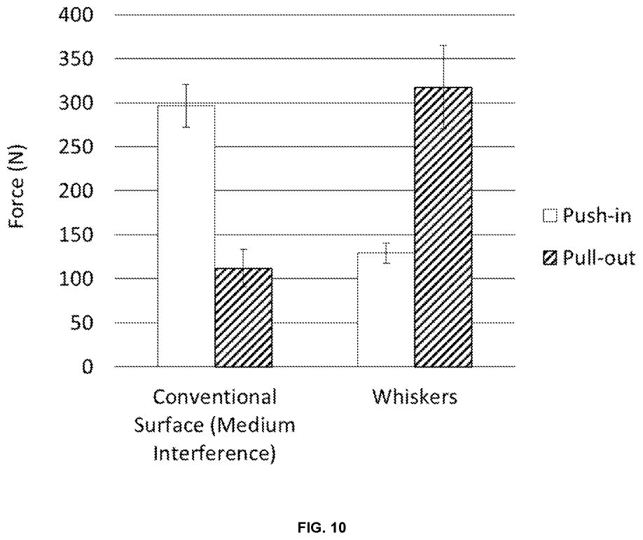

FIG. 9 is a side view of a surgical implant generally denoted 900. The surgical implant 900 comprises a body having a proximal and distal end, the body comprising a core 905, a plurality of whiskers 910 extending from the core 905 in a proximal direction, and a distal end portion 915. FIG. 9 is an embodiment of the invention where no lattice structure is present.

Embodiments of the present invention shown in FIGS. 1 to 9 highlight modifications to individual features of the invention which provide specific advantages to those particular embodiments of the invention. However, it would be equally apparent to the skilled person to consider combinations of these modifications to provide a stable surgical implant for the specific surgery being performed.

FIGS. 10 to 14 show the results of laboratory experiments to characterise the mechanical effectiveness of the present invention compared to conventional approaches. To evaluate the present invention, surgical implants were simplified to cylindrical specimens such as shown in FIGS. 1 and 2. Broadly speaking, the specimens were then subjected to push-in/pull-out tests with synthetic bone. Lower push-in forces would be beneficial as they would indicate reduced damage to the bone during implantation, meanwhile a larger pull-out force indicates greater anchoring into the bone and thus improved implant fixation. The most important finding of the experiments is shown in FIG. 10, where the push-in/pull-out forces of conventional implant surfaces are compared to those of the present invention. FIG. 10 shows that by using the hook-like whiskers of the present invention, reduced push-in forces and increased pull-out forces can be obtained in comparison to conventional implant surfaces.

A number of different design variations have been investigated including both the internal and external length of the whiskers, the thickness of the whiskers, the number of whiskers per row, the number of rows of whiskers, the aspect ratio of the surface, the shape of the whiskers, and the angle of the whiskers.

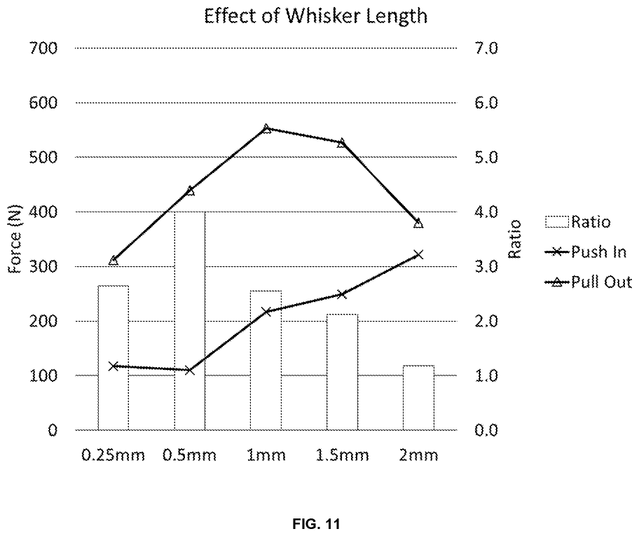

FIG. 11 shows the effect varying the length of whiskers has on the push-in and pull-out forces in an embodiment of the present invention. Specifically the effect of varying the external whisker length between 0.25 mm and 2 mm. The external whisker length of this embodiment refers to the length of whisker 110 projecting beyond the lattice structure 120, as measured normal to the surface of the core 105, and as best illustrated in FIG. 1. An external whisker length of 0.5 mm outside the lattice has the best push-in/pull-out ratio, whereas an external whisker length outside the lattice of 1.0 mm has the highest pull-out load. However, some whisker breakages were observed for larger whiskers so optimising the pull-out load, or the ratio of forces, is not the only consideration. If the whiskers are too short, no significant additional fixation over a traditional bone peg would be achieved; and if the whiskers are too long, there may be increased resistance to the surgical implant being inserted, and the whiskers may deform excessively or break during this process. The external whisker length of the surgical implant may be between 0.2 mm and 2.0 mm.

Preferably, the whiskers have an external length of 0.5 mm to 1.0 mm.

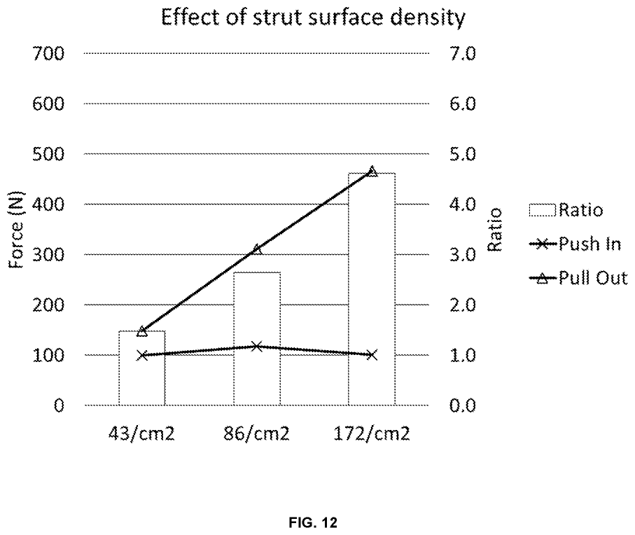

FIG. 12 shows the effect varying the density of the whiskers has on the push-in and pull-out forces on an embodiment of the present invention. Specifically, the effect of varying the whisker density between 43 whiskers/cm² to 172 whiskers/cm² has on push-in/pull-out forces of the specimens. Increasing the strut surface density for a given whisker length improves both the ratio and the max pull-out load. 172 whiskers/cm² is a maximum achievable density with a lattice structure, under current manufacturing constraints. The density of whiskers considered capable of achieving sufficient mechanical fixation may be as low as 25 whiskers/cm². However, without a lattice structure the whisker density could potentially increase to around 1000/cm².

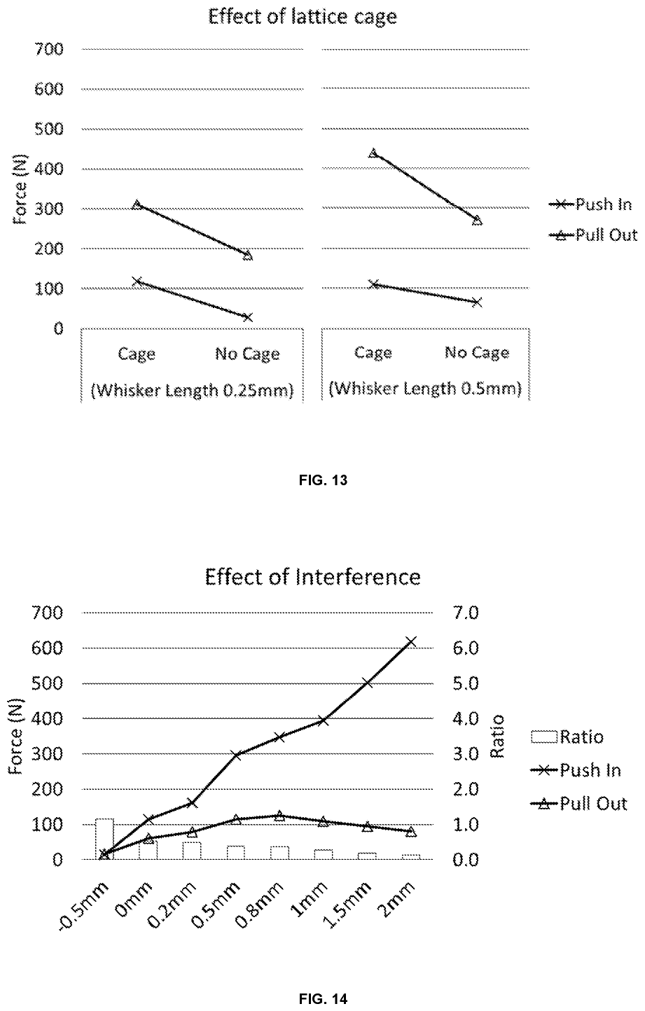

FIG. 13 shows the effect that having a lattice structure compared to not having a lattice structure has on the push-in and pull-out forces on an embodiment of the present invention. Specifically, the effect that introducing an outer layer of lattice set approximately 3 mm from the surface of the core has on push-in/pull-out forces of the specimens when the whisker lengths outside the lattice are respectively 0.25 mm and 0.5 mm. Including a lattice structure increased the pull-out load, but both designs with and without and outer cage have push-in/pull-out ratio's greater than one and hence could be beneficial compared to existing technology.

FIG. 14 shows, for comparison, the effect that varying the interference has on push-in/pull-out forces for conventional devices. Specifically, the effect that varying the interference between −0.5 mm and 2 mm has on push-in/pull-out forces of specimens that use existing technology. Increasing interference alone is not comparable to using the whisker design of the present invention, as there is no appreciable increase in pull-out force with increasing interference, but there is considerable increase in push-in force with increasing interference.

Embodiments of the invention may comprise a flat end portion at the distal end portion of the surgical implant. In some embodiments of the invention, the distal end portion may be tapered or chamfered. This aids insertion of the surgical implant into the bone structure.

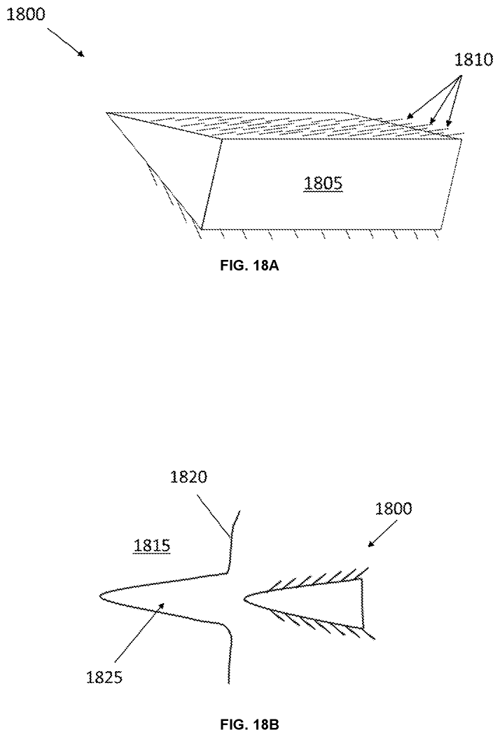

The surgical implant may be generally cylindrical, and take the form of a peg. However, it would be understood that other shapes would be considered appropriate so long as the implant could be inserted into the bone without causing undue damage to either the bone structure or the surgical implant, while providing sufficient mechanical fixation between the surgical implant and the bone structure. By way of example, the present invention may be applicable to a substantially wedge-shaped implant 1800, as shown in FIGS. 18A and 18B. As shown, the wedge-shaped implant 1800 comprises a body 1805 and a plurality of whiskers 1810 extending outwardly and pointing away from a bony surface 1820 of a bone section 1815 (see FIG. 18B). The whiskers 1810 of the wedged implant may be configured according to any of the other embodiments described in the present application. By way of example, such an implant would be suitable for use in opening wedge osteotomies, such as a high tibial osteotomy, or in spinal fusion cages.

The wedged implant 1800 may act as a spacer that is easy to insert into a bone section 1815 having a recess or crevice 1825. The whiskers 1810 will be arranged such that the implant 1800 will resist expulsion due to the in vivo forces generated during the bone healing process. Where traditional implants may be expelled from the recess 1825 by the in vivo forces, the mechanical interaction between the wedged implant 1800 and the bone surface 1820 of the bone section 1815 would secure the implant 1800 in place as the bone section 1815 heals.

Embodiments of the invention comprise whiskers of the surgical implant pointing towards the proximal end of the body and forming an acute angle with the body of the surgical implant. This provides reduced push-in force during implanting and increased pull-out force once the surgical implant has been implanted. Embodiments of the invention comprise whiskers an angle between 5° and 89° relative to the longitudinal axis of the body. Preferably, the whiskers are at an angle of 20° and 60° relative to the longitudinal axis of the body. The skilled person would equally consider that all whiskers need not be at the same angle and the arrangement of whiskers would depend on the desired mechanical interaction between the implant surface and the bone.

The whiskers of the surgical implant require a minimum thickness to provide sufficient stiffness to the whisker structure, dependent on the material from which the implant is manufactured. The whiskers may have a thickness between 100 μm and 1000 μm. Preferably, the whiskers have a thickness between 200 μm to 500 μm. It would be apparent to the skilled person that all whiskers do not need to be the same thickness, and that whiskers may have different thicknesses to achieve the desired overall mechanical interaction between the implant and the bone surface. Typically, the whiskers would have a length to thickness ratio in the range of 0.7 to 120, preferably in the range of 5 to 30.

A key feature of the design is that the whisker has to start deep inside the implant such that the overall length of the whisker is increased, whilst maintaining the same level of interference between the implant and bone. This design is only possible through an additive manufacturing process or 3D printing approach, as conventional manufacturing techniques such as forging or casting would not be able to create the required discrete whiskers originating from the core and protruding through voids in a lattice structure surrounding the core, as is achieved in the present invention. This is particularly advantageous given the recent trend for additive manufactured porous structures in orthopaedics. This allows for a range of materials suitable for additive manufacturing to be used, for example: titanium and alloys thereof, stainless steel, tantalum, and cobalt-chromium alloys. By way of example, a metal powder bed fusion additive manufacturing system able to manufacture parts in 50 μm layers from Titanium spherical powder is suitable for the manufacture of implants of the present invention. The titanium spherical powder may be Ti6Al4V ELI with a particle size ranging 10-45 μm, D50 of approximately 27 μm.

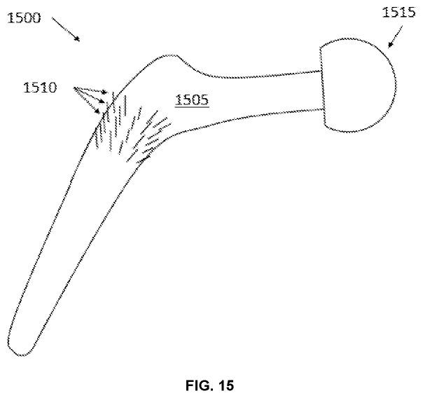

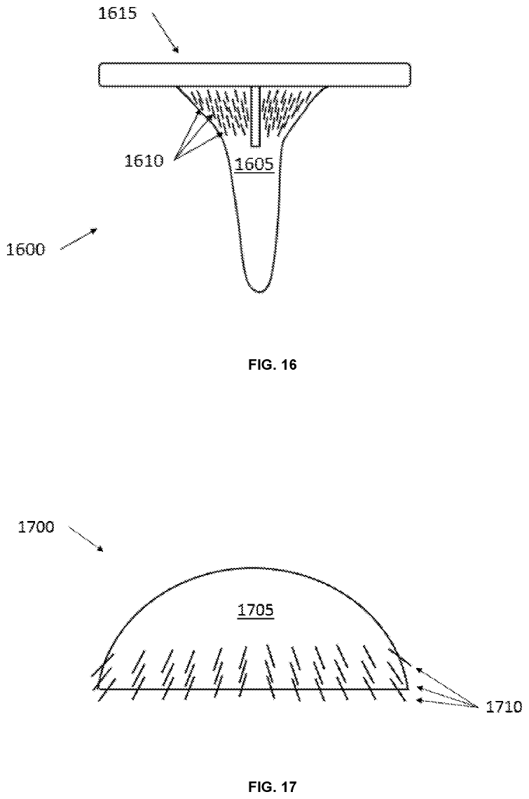

FIGS. 15 to 17 show examples of how the present invention can be applied to existing joint replacements to provide greater resistance to pull-out by incorporating whiskers on the implant body at the bone-implant interface. FIG. 15 shows a surgical implant surface according to an embodiment of the invention. The surgical implant surface has been applied to the stem of a replacement hip implant generally denoted 1500. The hip implant stem 1505 forms the core from which whiskers 1510 extend outwardly and point in a proximal direction towards femoral head 1515 of implant 1500. FIG. 16 shows a surgical implant surface according to an embodiment of the invention, applied to the keel of a tibial tray generally denoted 1600. The tibial keel 1605 forms the core from which whiskers 1610 extend outwardly and point in a proximal direction towards the tibial plateau 1615 of implant 1600. FIG. 17 shows a surgical implant surface according to an embodiment of the invention, applied to the bone-implant interface of an acetabular cup 1705. The acetabular cup 1705 forms the core from which whiskers 1710 extend outwardly. Whiskers 1710 form an acute angle with an axis normal to the surface of the acetabular cup 1705 originating from the corresponding joint between said whiskers 1710 and the acetabular cup 1705. Embodiments of the present invention applied to treat the surface of surgical implants 1500, 1600 and 1700 shown here are illustrative examples and it would be equally apparent to apply the present invention to other implant surfaces or replacement joints where mechanical stability between the implant and bone surface is required.

The structural characteristics of the embodiments shown in FIGS. 15 to 17 are substantially the same as those described in embodiments shown in FIGS. 1 to 9. However, it would be apparent to the skilled person that depending on the specific implant surface being modified, the whisker and lattice structures may be modified accordingly to achieve the level of mechanical fixation needed to result in a stable implant or replacement joint. Similarly, depending on the specific requirements of the surgery, some or all of the implant surface within a bone may be modified according to the present invention. Moreover, the thickness of the core may be modified so as to provide optimal physical properties, such as stiffness, for the implant as a whole, as well as providing a suitably-sized base from which the whiskers can extend so as to have optimum whisker length for a particular implant. By way of example, the thickness of the core, which is to say its diameter when the core has a round cross section or its largest lateral extent when non-round, may be determined on the basis of the desired overall whisker length, the desired extent of protrusion of the free end of the whisker beyond the outer extent of the lattice structure, and the desired depth of the lattice structure, as well as the size (diameter) of the hole in the bone into which the implant is to be inserted.

For an 8 mm diameter hole in a bone surface, for example, the outer diameter of a cylindrical peg for insertion in the hole may be selected to be 8 mm. Because the whiskers deflect on insertion, it is the outer diameter of the lattice structure that would be set at 8 mm. Thus, if the lattice is to have a depth of 3 mm, then the core would have a diameter (thickness) of 2 mm. The total length of the whiskers would be the depth of the lattice (3 mm) plus the desired amount of projection of the free end—say 0.2 mm—ergo 3.2 mm, all measurements made normal to the longitudinal axis. For such an implant for insertion into a bone hole of 8 mm diameter, the core may have a diameter of about 0.3 mm to 7.6 mm, preferably 2.9 mm to 6.0 mm. More generally, the diameter of the core (or the equivalent perimeter, for non-circular embodiments) may be in the range of 4% to 95%, preferably 36% to 75% of the total thickness of the implant, as measured normal to the longitudinal axis. The maximum total thickness of the implant in this context would typically be defined by the outer diameter of the distal end portion. Where there is a lattice structure surrounding the core, the maximum total thickness of the implant would typically be defined by the outer diameter of the lattice structure.

Of course, suitable changes may be made to account for varying thickness of the core along its length, as per the embodiment of FIG. 7. The size and shape of the core will be selected for suitability for the desired application of the implant. The requirements for an 8 mm bone anchor peg will be different to those of a total hip replacement femoral stem, which in turn would be different to those of an acetabular cup. By way of example, the diameter of a core for a total hip replacement femoral stem may be in the range of 6.4 mm to 45 mm, preferably 22 mm to 32 mm, and for an acetabular cup in the range of 29 mm to 73 mm, preferably 35 mm to 68 mm.

It is concluded that the invention could be used to improve initial implant stability either by increasing the anchoring force in bone for the same insertion force, or providing the same level of fixation with reduced insertion force. This defines a new set of rules for implant fixation using smaller low profile features, which are required for minimally invasive device design. The technology could be applied to any implant that needs to fixate into bone for example: total joint replacement, early intervention implants for cartilage repair, drug delivery ports in neurosurgery and dental implants, etc.

The present invention provides a surgical implant comprising a body having proximal and distal ends and a longitudinal axis extending therebetween, the body comprising a core and at least one end portion at the distal end and a plurality of discrete whiskers extending outwardly from the core and at an acute angle relative to a longitudinal axis of the body in a proximal direction. This invention exploits the increased design freedoms offered by additive manufacturing to make new fixation surfaces that use ‘whiskers’ to improve initial implant stability. These barb-like struts work by preferentially allowing movement in one-direction, whilst inhibiting the reverse movement. The technology could be applied to any medical device that a surgeon wishes to push into bone and then require it to not move from its implanted position post-operatively.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

1. A surgical implant, comprising:
a body having proximal and distal ends and a longitudinal axis extending therebetween,
the body comprising:
a core; and
at least one end portion at the distal end; and
a plurality of discrete whiskers extending outwardly from the core and at an acute angle relative to a longitudinal axis of the body in a proximal direction.

2. The surgical implant according to claim 1, wherein the end portion tapers distally.

3. The surgical implant according to claim 1 or claim 2, wherein the implant is substantially cylindrical.

4. The surgical implant according to claim 1 or claim 2, wherein the implant tapers distally.

5. The surgical implant according to claim 4, wherein the core has a constant cross-section perpendicular to the longitudinal axis and the whiskers are progressively shorter towards the distal end.

6. The surgical implant according to any of the preceding claims, wherein the density of whiskers over a surface of the core is in the range: 25 whiskers/cm² to 1000 whiskers/cm² and preferably 120 whiskers/cm² to 200 whiskers/cm².

7. The surgical implant according to any of the preceding claims, wherein the whiskers have a length in the range: 0.7 mm to 12.0 mm and preferably 2.5 mm to 6.0 mm, as measured normal to the surface of the core.

8. The surgical implant according to any of the preceding claims, wherein the angle between the whiskers and the longitudinal axis is in the range: 5° to 89° and preferably 20° to 60°.

9. The surgical implant according to any of the preceding claims, wherein some or all of the whiskers have a thickness in the range: 100 μm to 1000 μm and preferably 200 μm to 500 μm.

10. The surgical implant according to any of the preceding claims, wherein some or all of the whiskers are tapered in thickness along all or part of their length.

11. The surgical implant according to any of the preceding claims, wherein the whiskers are tapered in thickness along the length of the implant, such that the whiskers at the distal end are progressively thinner than the whiskers at the proximal end.

12. The surgical implant according to any of the preceding claims, wherein the core is tapered, narrowing towards the distal end.

13. The surgical implant according to any of the preceding claims, wherein some or all of the whiskers have a forked end.

14. The surgical implant according to claim 13, wherein the forked end is bifurcated and wherein each branch of the bifurcation lies in a plane outwardly offset from and substantially tangential to the surface of the core.

15. The surgical implant according to any of the preceding claims, wherein the core has a thickness in the range: 4% to 95% and preferably 36% to 75% of a total thickness of the implant, as measured normal to the longitudinal axis.

16. The surgical implant according to any of the preceding claims, further comprising a lattice structure surrounding the core.

17. The surgical implant according to claim 16, wherein the lattice structure has a depth in the range: 0.5 mm to 10 mm and preferably 2 mm to 5 mm, as measured normal to the surface of the core.

18. The surgical implant according to claim 16 or claim 17, wherein the lattice structure has substantially the same outer perimeter as the outer perimeter of the distal end portion of the body.

19. The surgical implant according to any of claims 16 to 18, wherein the lattice structure comprises a plurality of elements interconnected at nodes.

20. The surgical implant according to any of claims 16 to 19, wherein the plurality of elements form voids with multiple vertices.

21. The surgical implant according to any of claims 16 to 20, wherein the lattice is formed by at least one layer of elements.

22. The surgical implant according to any of claims 16 to 21, wherein the whiskers each extend through a respective void defined within the lattice structure.

23. The surgical implant according to claim 22, wherein the whiskers each extend beyond the lattice structure by a distance in the range: 0.2 mm to 2.0 mm, and preferably 0.5 mm to 1.0 mm, as measured normal to the surface of the core.

24. The surgical implant according to any of claims 16 to 23, wherein the whiskers are arranged to come into contact with a lower cage element defining the associated void when the whiskers are urged to deflect downwardly relative to the core.

25. The surgical implant according to any of claims 16 to 24, wherein the whiskers are arranged to avoid contact with any cage element when the whiskers are urged to deflect upwardly relative to the core.

26. The surgical implant according to any of the preceding claims, wherein the surgical implant is additively manufactured.

27. The surgical implant according to any of the preceding claims, wherein the surgical implant comprises one or more of the following materials: titanium and alloys thereof, stainless steel, tantalum, and cobalt-chromium alloys.

28. The surgical implant according to any of the preceding claims, wherein the implant comprises a surgical anchor or a surgical peg, or comprises part of a larger surgical implant.

29. A surgical implant surface comprising:
a body having a plurality of discrete whiskers extending from the surface of the body,
wherein an angle between each of one or more of the whiskers and a respective axis normal to the surface of the body originating from a corresponding joint between each respective whisker and the body is in the range: 5° to 89° and preferably 20° to 60°.

30. The surgical implant surface according to claim 29, wherein the density of whiskers is in the range: 25 whiskers/cm² to 1000 whiskers/cm² and preferably 120 whiskers/cm² to 200 whiskers/cm².

31. The surgical implant surface according to claim 29 or claim 30, wherein the whiskers have a length in the range: 0.7 mm to 12.0 mm and preferably 2.5 mm to 6.0 mm, as measured along the respective normal axis.

32. The surgical implant surface according to any of claims 29 to 31, wherein the whiskers have a thickness in the range: 100 μm to 1000 μm and preferably 200 μm to 500 μm.

33. The surgical implant surface according to any of claims 29 to 32, wherein some or all of the whiskers are tapered along all of part of their length.

34. The surgical implant surface according to any of claims 29 to 33, wherein the whiskers are tapered in thickness across the surface, such that the whiskers at one end of the surface are progressively thinner than the whiskers at an opposite end of the surface.

35. The surgical implant surface according to any of claims 29 to 34, wherein some or all of the whiskers have a forked end.

36. The surgical implant surface according to any of claims 29 to 35, further comprising a lattice structure covering some or all of the surface.

37. The surgical implant surface according to any of claim 36, wherein the lattice structure comprises a plurality of elements interconnected at nodes.

38. The surgical implant surface according to claim 36 or claim 37, wherein the plurality of elements form voids with multiple vertices.

39. The surgical implant surface according to any of claims 36 to 38, wherein the lattice is formed by at least one layer of elements.

40. The surgical implant surface according to any of claims 36 to 39, wherein each whisker extends through a respective void defined within the lattice structure.

41. The surgical implant surface according to claim 40, wherein the whiskers each extend beyond the lattice structure by a distance in the range: 0.2 mm to 2.0 mm, and preferably 0.5 mm to 1.0 mm, as measured along the respective normal axis.

42. The surgical implant surface according to any of claims 36 to 41, wherein the whiskers are arranged to come into contact with a lower cage element defining the associated void when the whiskers are urged to deflect towards said normal axis.

43. The surgical implant surface according to any of claims 36 to 42, wherein the whiskers are arranged to avoid contact with any cage element when the whiskers are urged to deflect away from said normal axis.

44. The surgical implant surface according to any of claims 29 to 43, wherein the surgical implant surface is additively manufactured.

45. The surgical implant surface according to any of claims 29 to 44, wherein the surgical implant surface comprises one or more of the following materials: titanium and alloys thereof, stainless steel, tantalum, and cobalt-chromium alloys.

46. The surgical implant surface according to any of claims 29 to 45, wherein the surface comprises part of a larger surgical implant having a longitudinal axis, proximal and distal ends, wherein a direction of insertion of the implant is in the distal direction, and wherein the surface is arranged such that the plurality of whiskers are at an acute angle relative to the longitudinal axis of the body in a proximal direction.
